(19) United States
(12) Patent Application Publication
(10) Pub. No.: US 2002/0156380 A1
(43) Pub. Date: Oct. 24, 2002

(54) RAMAN ENDOSCOPE

(76) Inventors: MICHAEL S. FELD, WABAN, MA (US); JOSEPH BARAGA, SOMMERVILLE, MA (US)

Correspondence Address: THOMAS O HOOVER, HAMILTON BROOK SMITH AND REYNOLDS, TWO MILITIA DRIVE, LEXINGTON, MA, 021734799

(21) Appl. No.: 08/745,509

(22) Filed: Nov. 12, 1996

Related U.S. Application Data
(62) Division of application No. 08/144,782, filed on Oct. 29, 1993, now abandoned.

(51) Int. Cl. A61B 6/00; A61B 5/00

(52) U.S. Cl. 600/473; 600/477; 600/109; 600/130; 600/160; 250/332; 250/338.1; 250/341.8; 250/370.08; 250/370.14

(57) ABSTRACT

The invention relates to a Raman endoscope for diagnosing diseased tissue within the human body. An infrared sensitive array is used to form spectroscopy enhanced images of tissue where laser induced Raman scattering is used to identify and quantitatively measure constituents of diseased and healthy tissue.

### GOVERNMENT SUPPORT

Funding for research conducted in connection with the subject matter of the present application was provided under NIH Grant No. RR 02594.

### BACKGROUND OF THE INVENTION

In the United States heart attacks, almost entirely attributable to coronary atherosclerosis, account for 20-25% of all deaths. Several medical and surgical therapies are available for treatment of atherosclerosis; however, at present no in situ methods exist to provide information in advance as to which lesions will progress despite a particular medical therapy.

Objective clinical assessments of atherosclerotic vessels are at present furnished almost exclusively by angiography, which provides anatomical information regarding plaque size and shape as well the degree of vessel stenosis. The decision of whether an interventional procedure is necessary and the choice of appropriate treatment modality is usually based on this information. However, the histological and biochemical composition of atherosclerotic plaques vary considerably, depending on the stage of the plaque and perhaps also reflecting the presence of multiple etiologies. This variation may influence both the prognosis of a given lesion as well as the success of a given treatment. Such data, if available, might significantly assist in the proper clinical management of atherosclerotic plaques, as well as in the development of a basic understanding of the pathogenesis of atherosclerosis.

At present biochemical and histological data regarding plaque composition can only be obtained either after treatment, by analyzing removed material, or at autopsy. Plaque biopsy is contraindicated due to the attendant risks involved in removing sufficient arterial tissue of laboratory analysis. Recognizing this limitation, a number of researchers have investigated optical spectroscopic methods as a means of assessing plaque deposits. Such “optical biopsies” are nondestructive, as they do not require removal of tissue, and can be performed rapidly with optical fibers and arterial catheters. With these methods, the clinician can obtain, with little additional risk to the patient, information that is necessary to predict which lesions may progress and to select the best treatment for a given lesion.

Among optical methods, most attention has centered on ultraviolet and/or visible fluorescence. Fluorescence spectroscopy has been utilized to diagnose disease in a number of human tissue, including arterial wall. In arterial wall, fluorescence of the tissue has provided for the characterization of normal and atherosclerotic artery. However the information provided is limited by the broad line width of fluorescence emission signals. Furthermore, for the most part, fluorescence based methods provide information about the electronic structure of the constituent molecules of the sample. There is a need for non-destructive real time biopsy methods which provide more complete and accurate biochemical and molecular diagnostic information. this is true for atherosclerosis as well as other diseases which affect the other organs of the body.

### SUMMARY OF THE INVENTION

The present invention relates to vibrational spectroscopic methods using near-infrared and infrared (IR) Raman spectroscopy. These methods provide extensive molecular level information about the pathogenesis of disease. These vibrational techniques are readily carried out remotely using fiber optic probes or endoscopes. In situ vibrational spectroscopic techniques allow probing of the molecular level changes taking place during disease progression. the information provided is used to guide the choice of the correct treatment modality.

These methods include the steps of irradiating the tissue to be diagnosed with radiation in the infrared range of the electromagnetic spectrum, detecting light emitted by the tissue at the same frequency, or alternatively, within a range of frequencies on one or both sides of the irradiating light, and analyzing the detected light to diagnose its condition. Raman methods are based on the acquisition of information about molecular vibrations which occur in the rang of wavelengths between 3 and 300 microns. Note that with respect to the use of Raman shifted light, excitation wavelengths in the ultraviolet, visible and infrared ranges can all produce diagnostically useful information. In the Raman effect the spectral information occurs in the form of frequency components of returning light inelastically scattered by the molecules in the tissue. These frequency components are usually downshifted in frequency from that of the exciting light by the resulting frequencies of the scattering molecules. Note that the exciting light itself may be in the infrared, the visible or the ultraviolet regions.

Raman spectroscopy is an important method in the study of biological samples, in general because of the ability of this method to obtain vibrational spectroscopic information from any sample state (gas, liquid or solid) and the weak interference from the water Raman signal in the “fingerprint” spectral region. the system furnishes high throughput and wavelength accuracy which might be needed to obtain signals from tissue and measure small frequency shifts that are taking place. Finally, standard quartz optical fibers can be used to excite and collect signals remotely.

The present methods relate to infrared methods of spectroscopy of various types of tissue and disease including cancerous and pre-cancerous tissue, non-malignant tumors or lesions and atherosclerotic human artery. Examples of measurements on human artery generally illustrate the utility of these spectroscopic techniques for clinical pathology. In addition, molecular level details can be deduced from the spectra, and this information can be used to determine the biochemical composition of various tissues including the concentration of molecular constituents that have been precisely correlated with disease states to provide accurate diagnosis.

Another preferred embodiment of the present invention uses two or more diagnostic procedures either simultaneously or sequentially collected to provide for a more complete diagnosis. These methods can include the use of fluorescence of endogenous tissue, Raman shifted measurements.

A preferred embodiment of the present invention features a focal plane array (PFA) detector to collect NIR and or infrared Raman spectra of the human artery. One particular embodiment employs Nd:YAG laser light at 1064 nm to illuminate the issue and thereby provide Raman spectra having frequency components in a range suitable for detection by the CCD. Other laser emitting in the 1-2 micron wavelength range can also be used including Nd:Glass. Holmium:YAG, or infrared diode lasers, or other known lasers in the visible region. Other wavelengths can be employed to optimize the diagnostic information depending upon the particular type of tissue and the type and stage of disease or abnormality. Raman spectra can be collected by the FPA at two slightly different illumination frequencies and are subtracted from one another to remove broadband fluorescence light components and thereby produce a high quality Raman spectrum. The high sensitivity of the CCD detector combined with the spectra subtraction technique allow high quality Raman spectra to be produced in less that 1 second with laser illumination intensity described herein. One can also reduce or eliminate fiber fluorescence by collecting light above 800 nm and preferably between 1 and 2 microns.

In many clinical applications it is highly advantageous to obtain multi-pixel images from the tissue in order to survey larger regions and provide a geometrical layout of the tissue. This is particularly important when one is studying heterogeneous tissues and trying to identify focal regions of change, such as in dysplasia or atherogenesis. by using the Raman-scattered radiation to form images, we have a new opportunity to create maps of specific histochemical over a region of tissue.

The use of two-dimensional CCD arrays provides a natural means for spatially resolving the Raman signals. These systems provide for recording raman spectroscopic images from human tissue both in vitro and in vivo. Such imaging systems represent the important application of Raman spectroscopy and Raman histochemical analysis as a clinical tool.

A preferred embodiment includes NIR array detectors and tunable filters to provide Raman spectroscopic imaging systems. One embodiment includes a low spatial resolution (˜100 pixels) Raman imaging system, similar in concept to the present fiver optic prototype spectrograph/CCD system, which provides a complete Raman spectra for each pixel. A further embodiment a high resolution (˜10,000 pixels) Raman endoscopic imaging system for in vivo studies, based on use of a coherent fiber bundle, a tunable narrow band filter and a sensitive NIR two-dimensional array detector.

A preferred embodiment employs a low noise silicon CCD array detector with a good NIR sensitivity out to 1050 nm and high quality single-stage imaging spectrographs open possibilities for low spatial resolution NIR Raman spectroscopic imaging systems. This system provides Raman spectroscopic images from human artery tissue in vitro with our fiber optic spectrograph/CCD system using 850 nm excitation.

A sensitive IR focal plane array (FPA) detectors for both NIR Raman spectroscopy and imaging. These detectors utilize a variety of silicide Schottky-barrier and GeₓSi₁₋ₓ, heterojunction materials. They represent hybrid silicon CCD technology in which a thin layer of silicide material, platinum or palladium silicide, for example, is deposited on the detector surface, thus providing sensitivity in the 1-2 μm wavelength range and beyond. These detectors exhibit the extremely low read noise and, when cooled to 70-120° K., the extremely low dart current characteristic of silicon CCD devices. In the region of interest for NIR Raman spectroscopy of tissue, their quantum efficiency is in the range of 10-20%.

These IR sensitive FPA's provide great flexibility in using longer excitation wavelengths for NIR Raman studies. Specifically, by utilizing excitation wavelengths near 1064 nm, as in the FT/Raman system, fluorescence background will be negligible, dramatically reducing background counts. This will reduce the spectral noise, simplify and/or obviate the need for background substraction, and aid in detection of weak Raman bands. Also, in certain high resolution Raman imaging applications, only limited spectral regions will be available.

The present invention utilzes this wavelength flexibility further by measuring additional excitation wavelengths between 900 and 1500 nm. Schottky-barrier photodetector arrays are preferred for both NIR Raman spectroscopy and imaging in human tissue.

A further embodiment uses tunable acousto-optic filters for Raman imaging experiments. Tunable acousto-optic filters are now commercially available (Brimores Technology) in the NIR with large apertures (5×5 mm²), high spectral resolutions (25 cm⁻¹@900 nm), high efficiencies (80%), and wide spectral ranges (800-1800 nm). They can be computer controlled to access any given wavelength in under 1 ms. A filter of this type serves to replace the spectrograph for applications in which high spatial resolution images of one or a series of Raman bands is desired. The FPAs and associated filters are typically between 0.5 and 2 mm in diameter and can be placed at the distal end of the endoscope.

### BRIEF DESCRIPTION OF THE DRAWINGS

FIG. 1 is schematic illustrations of preferred systems for providing the spectroscopic measurements of the invention.

FIG. 2 illustrates a cross-sectional view of a preferred embodiment of the Raman endoscope of the present invention.

FIG. 3 illustrates a cross-sectional view of another preferred embodiemnt of the distal end of a Raman endoscope.

FIG. 4 illustrates a cross-sectional view of another preferred embodiemnt of the distal end of a Raman endoscope.

FIG. 5 illustrates a cross-sectional view of a Raman endoscope delivering broad band and laser radiation onto tissue and the collection of Raman scattered light from a known volume of tissue.

FIG. 6 includes NIR Raman spectra of (a) normal aorta (x8), (b) athermoatous plaque (x4), and (c)

FIG. 7 includes NIR Raman spectra of the structural proteins (a) elastin (bovine neck ligament), and (b) collagen (bovine achilles tendon, type I).

FIG. 8 includes NIR Raman spectra of proteoglycans (a) chondroitin sulfate A, sodium salt (bovine tracheae), and (b) hyaluronic acid, sodium salt (bovine tracheae).

FIG. 9 includes NIR Raman spectra of cholesterol and cholesterol esters known to be significant in atherosclerotic lesions. (a) Cholesterol; (b) cholesterol palmitate; (c) cholesteryl oleate: (d) cholesteryl linoleate.

FIG. 10 includes NIR Raman spectra of (a) oleic acid, (b) triolein, and (c) subtraction of the spectrum of cholesterol from cholesteryl oleate, (c) demonstrates that the major bands in the Raman spectrum of cholesteryl oleate is simply the sum of cholesterol plus oleic acid and the ester vibration at 1737 cm₋₁.

FIG. 11 includes NIR Raman spectrum of calcium hydroxyapatite.

FIG. 12 includes a plot integrated intensity ration of the 1440 cm⁻¹ band of cholesterol to 987 cm⁻¹ peak of Baˆ Sˆ O₄ vs. weight percentage of cholesterol in cholesterol:BaSO₄ mixture (the symbols in the axes labels are as defined in eqn. (2) in the test). The slope of the line is 2.72; the regression coefficient is 0.997.

FIG. 13 is an NIR Raman spectra of (a) cholesterol, and (b) 50:50 by weight cholesterol: BaSO₄ mixture.

FIG. 14 includes measured Raman spectrum of 50% protein (25% collagen, 25% elastin) 50% lipid (25% cholesterol, 12.5% cholesteryl oleate, 12.5% cholesteryl linoleate) mixture, along with model calculated fit and residual.

FIG. 15 includes a plot of component weight percentages calculated from model vs. measured weight percentages. (a) Total protein (collagen+elastin). The slope of the line is 0.94; the regression coefficient is 0.98. (b) Total lipid (cholesterol+cholesteryl oleate+cholesteryl linoleate). The slope of the line is 0.94; the regression coefficient is 0.98.

FIG. 16 includes a plot of component weight percentages calculated from model vs. measured weight percentages. (a) Cholesterol. The slope of the line is 1.08; the regression coefficient is 0.98. (b) Total cholesterol ester (cholesteryl oleate+cholesteryl linoleate). The slope of the line is 0.81; the regression coefficient is 0.97.

FIG. 17 includes a plot of component weight percentages calculated from model vs. measured weight percentages. (a) Cholesteryl oleate. The slope of the line is 0.64; the regression coefficient is 0.93; (b) Cholesteryl linoleate. The slope of the line is 0.98; the regression coefficient is 0.93.

FIG. 18 includes a plot of component weight percentages calculated from model vs. measured weight percentages. (a) Collagen. The slope of the line is 1.21; the regression coefficient is 0.89. (b) Elastin. The slop of the line is 0.68; the regression coefficient is 0.73.

FIG. 19 includes a measured Raman spectrum of normal aorta, along with model calculated fit and residual (The negative spike at 1500 cm⁻¹ is due to spurious noise.)

FIG. 20 includes measured Raman spectrum of atheromatous plaque, along with model calculated fit and residual.

FIG. 21 includes measured Raman spectrum of calcified atheromatous plaque (exposed calcification), along with model calculated fit and residual. The residual has been offset from zero for clarity.

### DETAILED DESCRIPTION OF PREFERRED EMBODIMENT

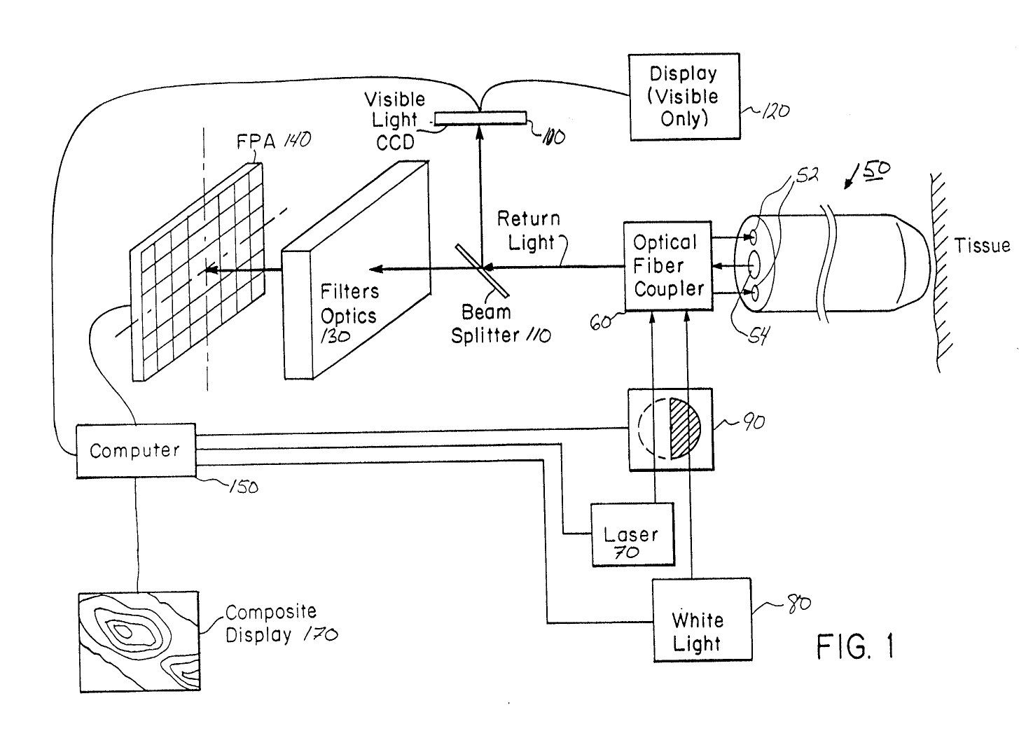

FIG. 1 illustrates a system for spectrally resolving spatial images of tissue which is constructed according to the principles of the present invention. Specifically, a distal end of a laser endoscope 50 is placed in close proximity to tissue which a user intends to spectroscopically analyze. This object tissue is illuminated by infrared or visible wavelength electromagnetic radiation conveyed by source fibers 52 contained in the laser endoscope 50. Radiation reflected from the tissue is captured by a collection bundle 54 and conveyed through a flexible catheter body to a proximal end, which is mated to a fiber optic coupler 60.

The fiber optic coupler 60 merges radiation received from a Nd:YAG laser 70 or a visible light endoscope 80 into the source fibers 52 of the laser catheter 50. The Nd:YAG laser 70 generates infrared radiation having a wavelength of approximately 1.06 μm. Since the one micrometer laser light is used for excitation, the problems associated with background fluorescence is negligible, substantially reducing background counts. This will reduce spectral noise, simplify and/or obviate the need for background subtraction, and aid in detection of weak Raman bands.

The visible light generator 80 can be a white light source such as a halogen lamp. This generator enables visual imaging of the object tissue to take place simultaneously or almost simultaneously with the Raman spectroscopy provided by the infrared radiation.

Two sources of light are alternatively blocked by an intervening half-moon shutter device 90 so that the object tissue will illuminated by either the visible light or the infrared light at any one moment. Alternatively, the computer can electronically switch between Nd:YAG laser 70 and the visible light generator 80 to ensure that the sources are not simultaneously active.

The fiber optic coupler 60 also couples the return radiation received from the collection bundle 54 to a beam splitter 110. The beam splitter 110 splits the return radiation into two beams. A first beam is focused on a charge coupled device 100. Since this charge coupled device 100 is only sensitive to the visible wavelengths of light, the resulting electrical signal will be representative of the visible light image of the object tissue illuminated the visible light generator 80. The visible light image signal is encoded and provided to both a visible light display device 120 which generates a video image of the object tissue and to the computer 150.

The second beam of the return radiation from the beam splitter 110 is provided to tunable acousto-optic filters 130. These filters have wide spectral ranges 800 to 1,800 nm and are capable of accessing any given wavelength within their respective spectral ranges within approximately 1 ms. Additionally, the filters 130 can have large apertures of approximately five millimeters square or smaller as needed, high spectral resolutions of 25 cm⁻¹ at 900 nm, and efficiencies of approximately 80%. Filters capable of meeting these criteria are manufactured by Brimrose Technology and are commercially available.

The second beam filtered by the acousto-optic filters 130 is then imaged on an infrared radiation Focal Plane Array FPA detector 140. This FPA detector utilizes a variety of silicide Schottky-barrier and GeₓSi₁₋ₓ heterojunction materials. They represent a hybrid silicon CCD technology in which a thin layer of silicide material, preferably platinum or palladium silicide, is deposited on a detector surface, thus providing a sensitivity in the 1-2 μm wavelength range and beyond. These types of detectors exhibit an extremely low read noise and, when cooled below 70 to 120 K, the extremely low dark current characteristic of silicon CCD devices. In the range of interest, their quantum efficiency is between 10 and 20%. Therefore, when the tunable acousto-optic filters 130 are tuned to the band of interest, the full 2-dimensional structure of the FPA detector 140 is utilized for image formation.

The FPA detector 140 converts the filtered second beam into an electrical signal which is representative of the infrared image of the object tissue. This infrared imaging signal is encoded and provided to the computer 150 along with the visible light image signal generated by the charge coupled device 100. This computer 150 performs Raman spectral analysis and enhancement of the infrared imaging signal and then selectively mixes spectrally enhanced signal with visible imaging signal to generate a combined signal. This combined signal is displayed on a second diagnostic display device 170 thus providing a composite display including both topographic information arising from of the visible light imaging and histochemical information from the infrared imaging in the form of a contour map.

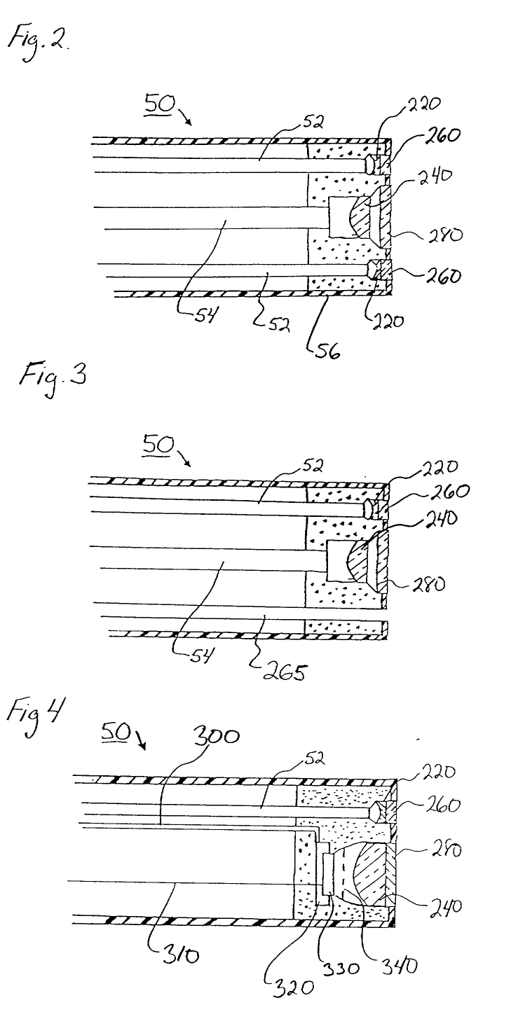

FIG. 2 illustrates the distal end of the laser catheter 50. At this distal end, a collection bundle 54 is centrally located along the axis of the laser endoscope 50. Source fiber lenses 220 are positioned in front of the source fibers 52 to disperse the light so that the object tissue is evenly illuminated within the collection bundle's field of view. A collection bundle lens 240 in front of the collection bundle forms an image of the object tissue on the terminal end of the collection bundle. Each of the sources Fiber lenses and the collection bundle lens are protected by transparent windows 260 and 280 which mate Flush with the catheter housing 56.

An second embodiment illustrated in FIG. 3 provides a biopsy channel 265 along the length laser endoscope 50. This is a two way channel that both enables tissue samples to be extracted and the injection of air or water to clear any debris from the transparent windows 260, 280.

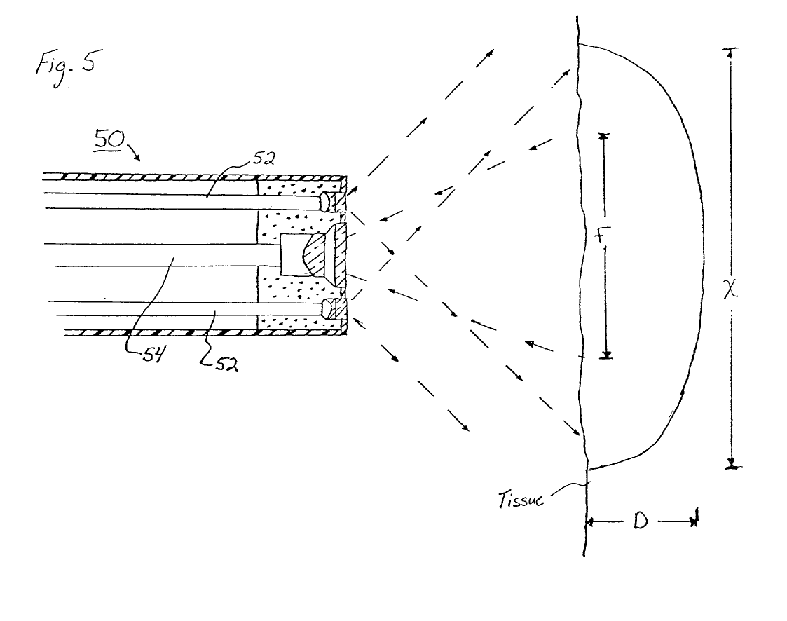

FIG. 4 illustrates a third alternative embodiment in which the FPA detector 140 is positioned in the distal end of the laser endoscope. Since the FPA detector 140 is provided without the intervening collection bundle, the full spatial resolution of the FPA detector 140 can be realized. A lens 240 is provided so that an image is formed on the FPA detector while an optical filtering device 340, such as an acousto-optic filter, is positioned between lens 240 and the FPA 140 to enable isolation of the spectral bands of interest. Power to the FPA detector 140 and signals representing the detected images are transmitted by cable 310. Since the FPA detector must be colled for proper operation, it is set in a heat sink 320 which receives collant from line 300.

FIG. 5 illustrates the field of view of the collection bundle 54 compared with the region of the tissue illuminated by the source fibers 52. The region of substantial illumination, x, is larger than the portion of the tissue within the collection bundle's field of view, f, so that an even distribution of light within the field is obtained. FIG. 5 also illustrates that the tissue is illuminated to a depth D. The depth of illumination is a factor in the spectral analysis since the received Raman spectra includes a portion arising out of the sub-surface excitation.

For single pixel measurements a Perkin-Elmer Fourier transform infrared spectrometer can be utilized for NIR FT Raman spectroscopy where the Raman accessory employs a 180° back-scattering geometry and a cooled (77 K) InGaAs detector. This system is described in applications incorporated elsewhere herein by reference. A 1064 nm CW ND:YAG laser was used for exciting samples, with 400 nm W laser power in a 1 mm diameter spot on the sample. Spectra of components are the sum of 256 scans recorded at 8 cm⁻¹ resolution (approximately 18 min collection time), and those of tissues are the sum of 512 scans recorded at 8 cm⁻¹ resolution (35 min collection time). For multi-pixel high speed diagnostics and imaging the infrared CCD sensors described above are utilized.

This system can be used in conjunction with diagnostic and treatment systems described in more detail in U.S. Pat. No. 5,125,404, and in U.S. Ser. No. 08/107,854 filed on Aug. 26, 1993 which is identical to International application No. PCT/US92/003-420, the contents of which are all incorporated herein by reference.

FPA arrays operating in the infrared in the following publications, Cautella, “Space Surveillance With Medium-Wave Infrared Sensors”, The Lincoln Laboratory Journal, Volume 1, Number 1 (1988), Kosonocky et al, “Design, Performance and Application of 160×244 Element IR-CCD Imager”, Proc. 32nd National Infrared Information Symp. 29, 479 (1984) and Taylor et al., “Improved Platimum Silicode IRCCD Focal Plane” SPIE 217,103 (1080) all which are incorporated herein by reference.

To extract quantitative histochemical information, relative Raman cross-sections were measured by using BaSO₄ as a Raman scattering internal intensity standard, and the behavior of the raman signals of individual biomolecules with concentration was explored. Mixtures of a known weight percent of the powder of the compound of interest and BaSO₄ were finely ground using a mortar and pestle until they visually appeared to be homogenized, and then placed in a fused silica cuvette. For each sample, at least three measurements were made by irradiating different spots on the sample; the variation in the cross-section values was within ±15%. Since, no polarization analyzer was employed, the weight cross-sections derived here represent the sum of the scattering contributions from both perpendicular and parallel polarizations. Mixtures of tissue components themselves, without BaSO₄, were also examined both a powders and as saline slurries.

Human aorta was chosen for initial study as an instance of atherosclerotic artery tissue. Samples were obtained at the time of postmortem examination, rinsed with isotonic saline solution (buffered at pH 7.4), snap-frozen in liquid nitrogen, and stored at −85° C. until use. Prior to spectroscopic study, samples were passively warmed to room temperature while being kept moist with the isotonic saline. Normal and atherosclerotic areas of tissue were identified by gross inspection, separated, and sliced into roughly 8×8 mm² pieces of known thickness. The tissue samples were placed in a suprasil quartz cuvette with a small amont of isotonic saline to keep the tissue moist, and one surface in contact with the window was irradiated by the laser. After spectroscopic examination, all specimens were histologically analyzed to verify the gross identifications.

To quantify the observed spectral signals from human artery, the first question which must be addressed is the choice of the biological substituents which should be examined. Normal human artery is composed of three distinct layers: intima, media and adventitia. The intima, normally 50-300 μm thick depending on the artery, is the innermost layer. It is mainly composed of collagen fibers and ground substance, primarily formed from proteoglycans. A single layer of endothelial cells in the vessel lumen protects the intima from injury. Normal intima is composed of up to 30% dry weight collagen (types I and III) and 20% elastin. The proteoglycans account for up to 3% of the dry weight. The media, several hundred microns thick, can be quite elastic or muscular depending on the artery. The structural protein elastin is the major component of aortic media, while smooth muscle cells make up the majority of the media in coronary artery. The outermost adventitial layer serves as a connective tissue network which loosely anchors the vessel in place, and is mainly made up of lipids, glycoproteins and collagen.

During the atherosclerotic process, the intima thickens due to collagen accumulation and smooth muscle cell proliferation, lipid and necrotic deposits accumulate under and within the collagenous intima, and eventually calcium builds up, leading to calcium apatite deposits in the artery wall. Collagen can account for up to 60% of the dry weight of the atherosclerotic intima, and lipids can account for up to 70% depending on the lesion type. Elastin is generally less than 10% and the ground substance is equivalent to that found in normal intima. The lipids in the atherosclerotic lesion are primarily composed of cholesterol and cholesterol esters, with cholesteryl palmitate, cholesteryl oleate and cholesteryl linoleate accounting for up to 75% of the cholesterol esters.

These considerations suggest that the primary species are collagen, elastin, cholesterol, the cholesterol esters of palmitic acid, oleic acid and linoleic acid, and calcium hydroxyapatite. The proteoglycans are also measured and can contribute to diagnostic evaluation.

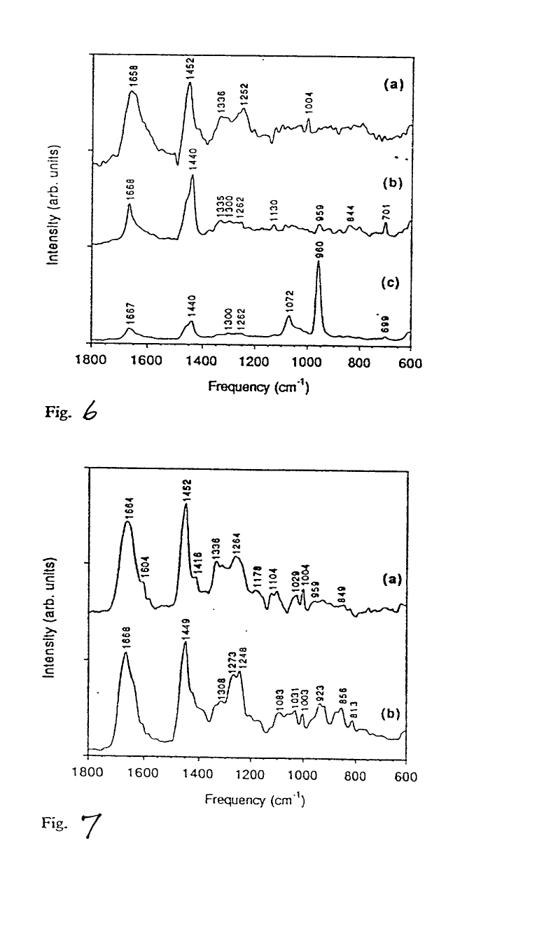

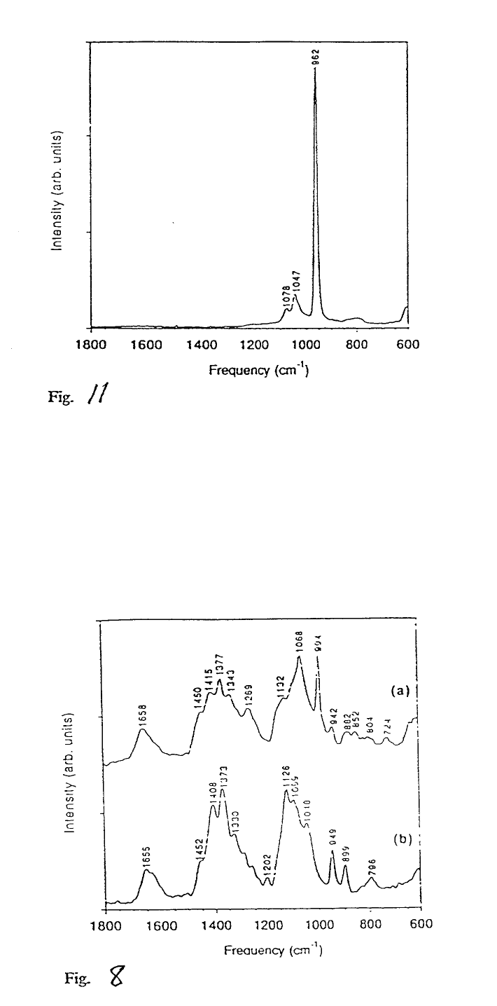

FIG. 6 shows the NIR Raman spectra obtained from typical specimens of normal, atheromatous and calcified human aorta. As demonstrated by comparing FIG. 6A with the spectra of elastin (bovine neck ligament) and collagen type I (Bovine achilles tendon) (FIG. 7), the spectrum of normal aorta is dominated by bands due to the proteins. In particular, the bands observed at 1658 and 1252 cm⁻¹ can be assigned to amide backbone vibrations, while the peak at 1452 cm⁻¹ is due to C-H bending of the protein. Note that bands due to proteoglycans, such as chondroitin sulfate A and hyaluronic acid (FIG. 8), which are known to make up the ground substance in artery wall, do not appear to contribute significantly to the spectra, as might be expected from their low concentrations.

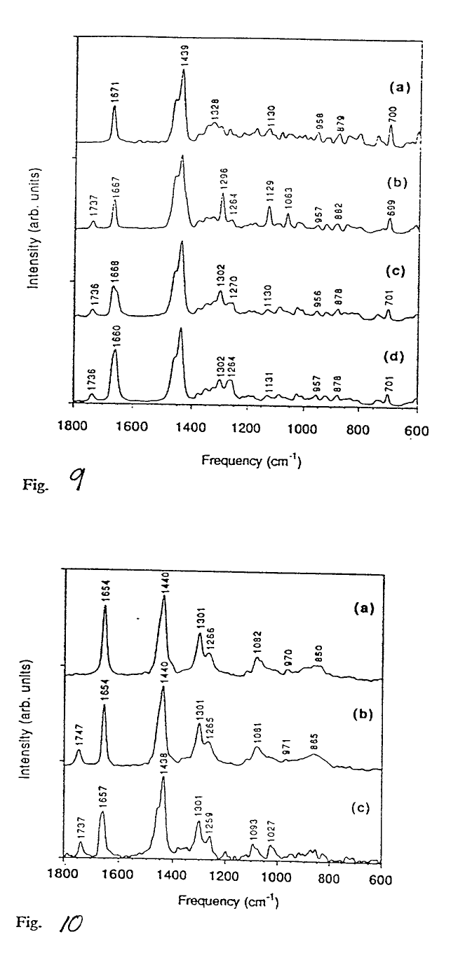

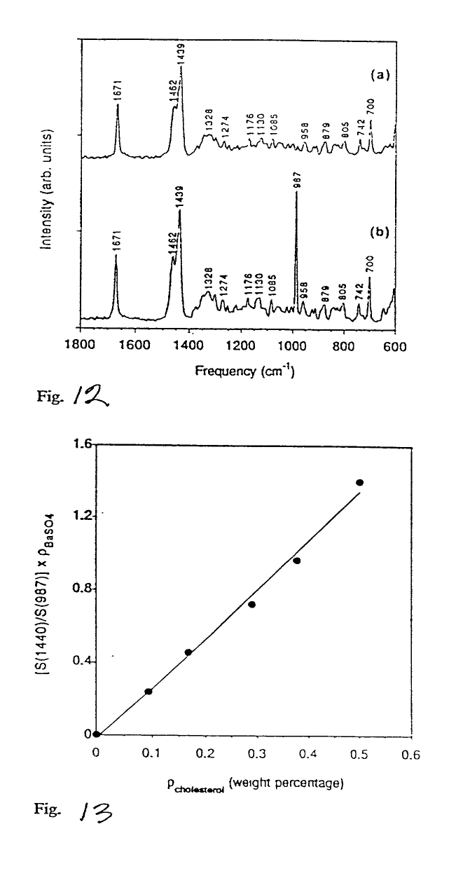

The spectrum of the atheromatous plaque (FIG. 6b) is distinctly different from that of normal aorta (FIG. 6a). In particular, there are many more bands in the atheromatous plaque spectrum below 1000 cm⁻¹. Consideration of the physiology of these plaques, as discussed above, and comparison of the spectra with several of the predominant cholesterol esters shown in FIG. 9 indicate that many of the bands in these spectra are due to cholesterol and its esters. In fact, the band at 700 cm⁻¹, due to the sterol ring, appears to serve as a marker for the existence of cholesterols in atherosclerotic lesions, while the other bands can be used to separate the various contributions of the esters to the spectrum. Some of the bands in the spectra of the cholesterol esters can be directly attributed to the spectra of the fatty acid side chains. This is demonstrated in FIG. 10c, where the spectrum of cholesterol is subtracted from cholesteryl oleate. The result is a spectrum nearly identical to that found in FIG. 10a of oleic acid, with the exception of the ester vibrational band at 1737 cm⁻¹. This also points out the ability of the Raman method to distinguish between triglycerides (glycerol triesters), which have an ester frequency around 1737 cm⁻¹ (FIG. 10b), and the cholesterol esters which have ester vibrational frequencies around 1737 cm⁻¹.

The NIR Raman spectra of calcified plaques (FIG. 6c) have additional bands at 960 and 1070 cm⁻¹. Comparison of calcified plaque spectra with the NIR Raman spectrum of calcium hydroxyapatite (FIG. 11) indicates that this salt is the primary contributor to the 960 cm⁻¹ band. However, the 1070 cm⁻¹ band seen in calcified plaque may have a large contribution from carbonate apatite (see below).

Having established the identity of the major contributors to the NIR Raman spectra of artery, we now utilize the Raman spectra to extract quantitative biochemical information. In a preferred embodiment two pieces of information are employed. First, the Raman scattering cross-section for each of the species must be measured relative, to a standard, so that meaningful comparison between bands of different molecules can be carried out. Secondly, the behavior of the Raman signals with respect to concentration in a highly scattering medium such as tissue must be measured.

In order to address the first issue, we measured the integrated Raman intensities from the bands of many compounds known to be important in atherosclerotic tissue. As discussed in Section 2, the band intensities were studied in BaSO₄ powder mixtures in order to utilize the strong SO₄²⁻ band at 987⁻¹ as an internal reference standard. For a given intensity, I₀ (W cm⁻²) and collection time, t (s), the integrated Raman signal in W for a band at a frequency νᵢ, S(νᵢ) measured at the detector is given by

$$S ( {v}_{i} ) = {ηI}_{o} t ξ l {ϱ ( \frac{\partial σ}{\partial Ω} )}_{vi} \qquad ( 1 )$$

where η is the detector quantum efficiency (electrons/Photon) and ξ is the efficiency of the optical system. The instrument throughput, θ (cm² sr), is given by the product of the collection area, A (cm²), and the solid angle of collection, Ω (sr), and the sampling length, l (mm), is primarily determined by the collection optics. ρ is the concentration in either g cm⁻³ or molecules cm⁻¹; for the former concentration units (∂σ/∂Ω)νᵢ is a weight Raman cross-section (cm² (g·sr)⁻¹) while for the latter it is a molecular cross-section (sm² (molecule·SR)⁻¹).

η, I₀, t, ξ, ˆ N8,θ and l can be eliminated from consideration when using an internal standard. Comparing the BaSO₄ signal with the material of interest,

$$\frac{{( \frac{\partial σ}{\partial Ω} )}_{vi}}{{( \frac{\partial σ}{\partial Ω} )}_{{v}_{{BaSO}_{4}}}} = \frac{S ( {v}_{i} ) {ρ}_{{BaSO}_{4}}}{S ( {v}_{{BaSO}_{4}} ) ρ i} \qquad ( 2 )$$

We have ignored local field corrections for the local refractive indices in the condensed phase. In Table 1, we report the relative Raman weight cross-sections compared with 1 g BaSO₄ for several bands in collagen, elastin, cholesterol, the primary cholesterol esters (cholesteryl palmitate, cholesteryl oleate and cholesteryl linoieate), the triglyceride tripalmitin and its fatty acid side-chain palmitic acid. We have chosen to report the relative Raman weight cross-sections because for many biological components (e.g. elastin) the precise molecular weights are unknown.

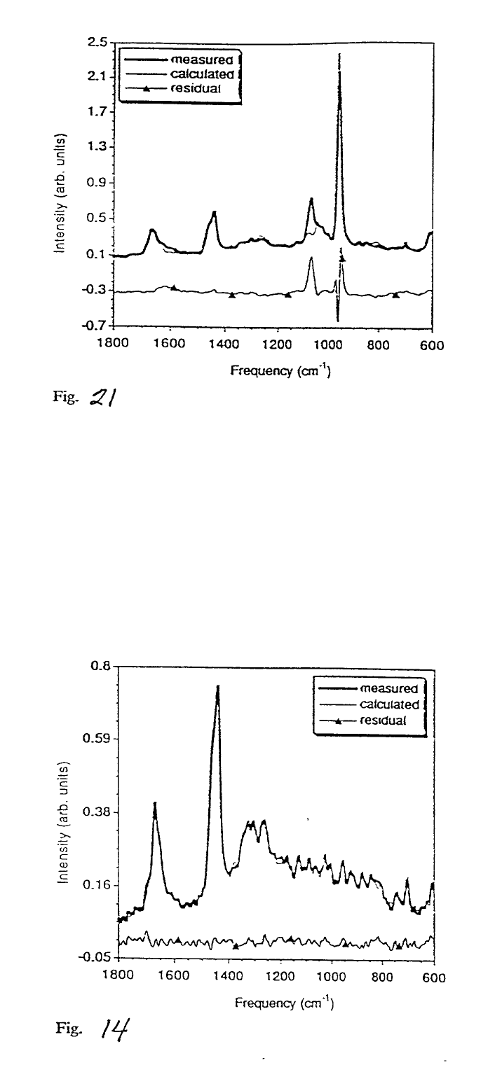

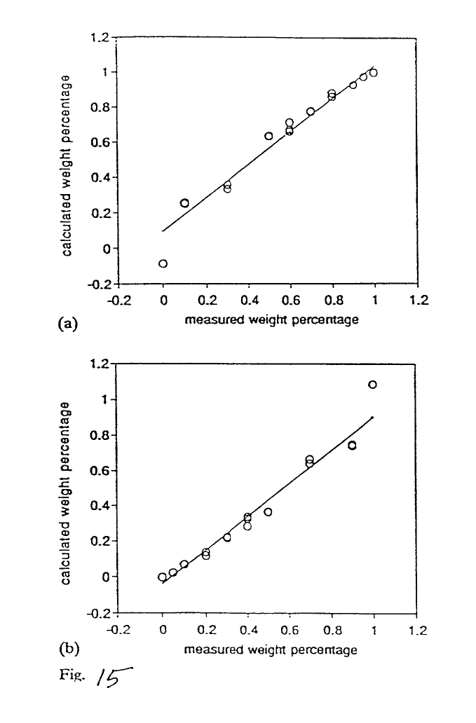

As an example, FIG. 12 shows the NIR FT Raman spectrum of a cholesterol: BaSO₄ powder mixture (50 wt. % cholesterol). In this experiment the CH₂ bending mode of cholesterol at 1440 cm⁻¹ is compared with that of the symmetric SO₄²⁻ stretching vibration of BaSO₄ at 987 cm⁻¹. The areas under each of the bands were determined and compared, yielding a relative Raman weight cross-section of 3.19. In order to test the linearity of the Raman signal in a highly scattering medium. The weight percentages of cholesterol and BaSO₄ were varied, and the integrated intensity ratio of the CH₋₂ bending mode of cholesterol at 1440 cm⁻¹ to that of the BaSO₄ peak at 987 cm⁻¹ was measured. The plot of integrated intensity ratio versus weight percentage of cholesterol is shown in FIG. 13 and is found to be linear. The linearity of this plot is an indication of both the homogeneity of the powder mixture and the absence of any chemical interaction between the components of the mixture that cold alter the spectral features. The implication of this result is that apparently the tissue Raman spectra can be described in terms of a linear superposition of individual biochemical constituents as long as the specific scattering proprieties of tissue do not significantly distort the signal.

Having established the linear and chemical behavior of the powder mixtures with BaSO₄, the molecular Raman scattering cross-section of each given band for various lipids was estimate dosing BaSO₄ as a standard (Table 2). In doing this, we utilize the relative weight cross-sections listed in Table 1, the known molecular weights of these compounds, and the value of the Raman cross-section of BaSO₄ reported in the literature. For given cholesterol lipid, the scattering cross-section for —CH₂ bending vibrations is high than other modes. The molecular Raman cross-section (Table 2) of the CH₂ bending modes of cholesterol with the additional fatty acid side-chains in the case of esters. The increase in this value for cholesteryl oleate (C18″1) and cholesteryl linoleate (C18:2) relative to cholesteryl palmitate (C16:0) is likely due to the increase in the number of —CH₂ groups in the side-chain. The degree of unsaturation, or number of double bonds in the fatty acid side-chain, of the lipids is manifested in the molecular Raman cross-section values of the band around 1670 cm⁻¹. For example, cholesteryl palmitate, which like cholesterol has only one double bond in the ring, shows a molecular scattering cross-section of 0.77 relative to cholesterol. The molecular scattering cross-section of this same band in cholesteryl oleate, which has one ring and one side-chain double bond, is 2.58 times larger than that of cholesterol; in cholesteryl linoleate, with a total of three double bonds, this cross-section is 3.13 times larger than in cholesterol.

Both cholesterol and the cholesteryl lipids exhibit a unique Raman peak at 700 cm⁻¹ as a result of the steroid nucleus. Defining the molecular scattering cross-section for this mode in cholesterol to be 1.00, the relative molecular scattering cross-section value for this mode is decreased to nearly 0.55 in the cholesterol esters. This might be attributed to the substitution-induced effect on the ring skeletal mode. The ester band molecular scattering cross-section of tripalmitin is nearly four times higher than that of cholesterol esters, primarily because trapalimitin has three ester groups compared with the one in the cholesterol esters. Similarly, the relative molecular scattering cross-sections of all the modes of tripalmitin are nearly three times higher than those of palmitic acid. This is consistent with the molecular structure of tripalmitin, which is the triglyceride of palmitic acid.

For calcium hydroxyapatite, the weight scattering cross-section of the symmetric phosphate stretching mode, 0.36, is ten times greater than that of the anti-symmetric mode. In tissue, additional bands appear around the phosphate anti-symmetric stretching frequency, and thus the relative intensity of this band is larger. These bands are carbonated apatite as discussed below.

For equal weight percentage, the relative Raman cross-sections of lipid bands near 1440 cm⁻¹ are higher than those of protein and glycosaminoglycan modes. This suggests that if equal amounts (by weight) of lipids and proteins are present in a mixture, lipids are expected to contribute to the integrated area of —CH₂ bands nearly four times as much as proteins.

NIR FT Raman spectra of different biological components can qualitatively account for the observed features of the spectra of aorta. In addition, the signals behave in a linear fashion, even in the presence of a highly scattering medium such as BaSO₄.

A preferred procedure for analyzing the NIR Raman spectra is a simple linear superposition of the spectra of the biological substituents given by

R(ν)=Σχᵢrᵢ(ν)+poly3(ν) (3)

where R(ν) is the observed Raman spectrum of tissue, rᵢ(ν) is the Raman spectrum of the ith component normalized to a particular band, and χᵢ is the fir coefficient describing the spectral contribution of the ith component. Poly3(ν) is a third-order polynomial utilized to account for broad, featureless signals from tissue not accounted for by the basis set. In our procedure, the basis set of spectral lineshapes, rᵢ(ν), are given by the pure substance spectra (shown in FIGS. 7, 9 and 12), with the integrated intensity of the CH₂ bending band normalized to unity. The parameters χᵢ are determined using a linear least-squares fitting procedure. Using the relative Raman weight cross-sections of the Ch₂ band for the individual components determined above, the weight percentage wᵢ of each component can then be computed as follows:

$${w}_{i} = K = \frac{{𝒳}_{i}}{\frac{{( \frac{\partial σ}{\partial Ω} )}_{vi}}{( \frac{\partial σ}{\partial Ω} )}} \qquad ( 4 )$$

where K is determined by normalizing the sum of the weight percentages to unity. Alternatively, this can be written as

$${w}_{i} = \frac{\frac{{𝒳}_{i}}{{( \frac{\partial σ}{\partial Ω} )}_{vi}}}{\frac{{\sum }_{i } {χ}_{i}}{{( \frac{\partial σ}{\partial Ω} )}_{vi}}} \qquad ( 5 )$$

The Raman cross-section for the standard, BaSO₄, is not required to compute the weight percentages of individual components, as the weight percentages are measured relatively.

In order to initially test the capabilities of this approach, we measured FT Raman spectra of mixtures of the biological constituents with varying weight percentages. Each mixture spectrum was then fit to eqn. for R(ν), and the weight percentages calculated from eqn. for wᵢ were compared with the known weight percentages of the mixtures.

The analytical method has been applied to several specimens of normal and atherosclerotic aorta to examine the applicability of the basis set and to establish typical limits of sensitivity of this approach.

To evaluate the linearity of the raman signals, the limits of detection of important tissue constituents, and the accuracy of the process series of mixtures of the pure biological constituents were prepared with weight percentages that span the known compositions of normal and atherosclerotic artery. In the primary components of interest were those that play dominant roles in normal and atherosclerotic plaques: the proteins collagen and elastin, and cholesterol and cholesterol ester lipids.

Ten separate mixtures of protein and lipid were prepared, with varying protein/lipid weight percents ranging from 100% protein/0% lipid to 0% protein/1--% lipid. The protein portion consisted of collagen type I (bovine achilles tendon) and elastin (bovine neck ligament) in equal weight percentages (collagen:elastin-1:1), and the lipid portion consisted of equal weight percentages of cholesterol and cholesterol ester (cholesterol:cholesteryl oleate:cholesteryl linoleate=1:0.5:0.5). This range allowed evaluations of the accuracy of the linear representation for all five components and of detection limits for total protein and total lipid, as well as for the individual proteins and cholesterol lipids. Two consecutive Raman spectra were recorded from the same spot for each mixture to check the reproducibility in measurement, and Raman spectra from two separate spots wee recorded for two of the mixtures to check the homogeneity of the mixtures. Each Raman spectrum was then adjusted using eqn. (3) with the Raman lineshapes recorded from the five individual components. Each resultant fit coefficient χᵢ was then used along with the measured CH₂ band Raman weight cross-section of that component (listed in Table 1) to compute the weight percentage, wᵢ, for that component according to eqn. (4).

The Raman spectrum of the 50% protein (collagen 25%, elastin 25%) 50% lipid (cholesterol 25%, cholesteryl linoleate 12.5%) mixture is compared with the calculation in FIG. 14. The residual of the fit (also shown in FIG. 14) falls within the noise level of the spectrum, indicating a reasonable fit to the spectrum. The weight percentages calculated from the fit coefficients for this spectrum are protein 64% (collagen 26%, elastin 38%) and lipid 36% (cholesterol 20%, cholesteryl oleate 5%, cholesteryl linoleate 11%). Given the ±15% uncertainties in the measured Raman cross-sections and the inhomogeneities in the mixture, the calculated protein and lipid weight percentages agree with the measured percentages to within the experimental error. The differences among the individual protein and lipid component weight percentage calculated from the model and the measured weight percentages is primarily attributable to uncertainties in the cross-sections, along with uncertainties in the fit coefficients due to spectral noise (see below).

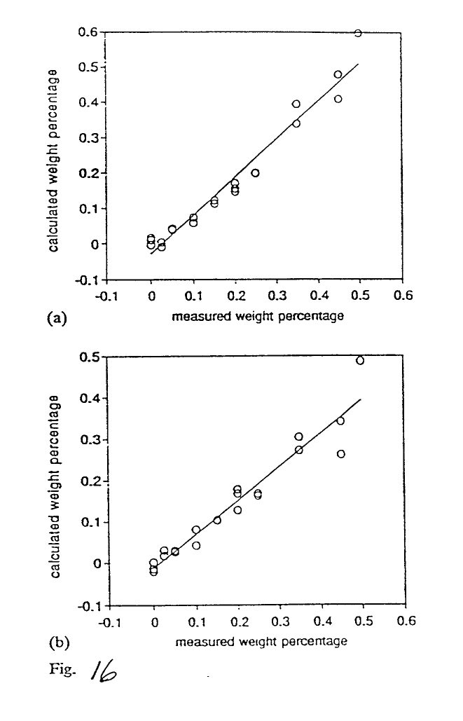

The weight percentages of total protein and total lipid calculated from the model are compared with the measured weight percentages in FIG. 15 for all the Raman spectra collected from the mixtures. These plots illustrate three important features regarding the calculated total protein and total lipid weight percentage. First, the calculated weight percentages are very linear over the entire range of mixture concentrations, supporting the validity of the linear representation. Second, a linear correlation between calculated and measured lipid weight percentages yields a slope of 0.94, which is essentially consistent with the expected value of 1. Any small discrepancy between this value and an exact match (slope=1) is attributable to systematic uncertainties from two sources. One source is the difficulty in achieving completely homogeneous mixtures due to differences in the physical properties of the components. For example, collagen, elastin and cholesterol are powdery and cholesterol oleate and linoleate are pasty. The other source of systematic uncertainty derives from measurement errors in Raman cross-section values, which propagate in the calculation of wight percentages. Third, uncertainties in the calculated weight percentages due to spectral noise, which are illustrated by the scatter of the data points about the linear correlations in FIG. 15, are relatively small. These uncertainties determine the detection limits for lipid and protein; the data in FIG. 15 indicate that these limits are 5% or less for total lipid and 10-15% for protein. The difference in detection limits between protein and lipid are in large part due to the three-fold smaller CH₂ band Raman weight cross-sections for proteins (see Table 1).

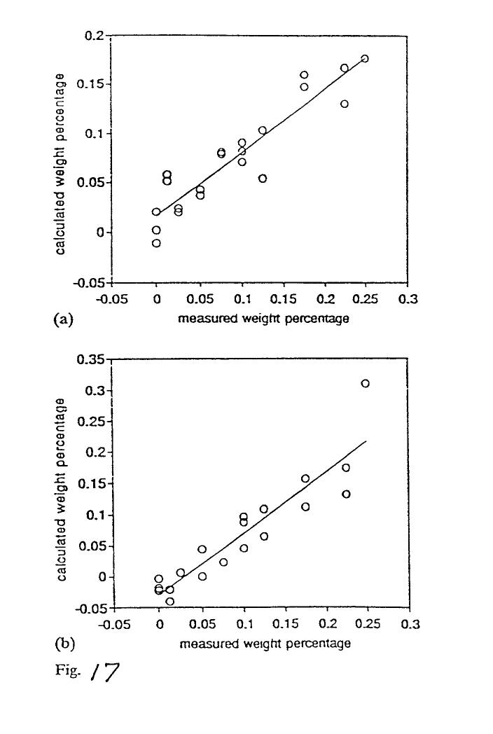

At finer level of detail, the lipids can be divided into cholesterol and cholesterol esters. Cholesterol and total cholesterol esters (oleate=linoleate) weight percentages determined form the Raman spectra are compared with the directly measured weight percentages in FIG. 16. the individual cholesterol ester (oleate, linoleate) weight percentages are plotted in FIG. 17. In all cases, the calculated and measured weight percentages appear to be linearly correlated to within the parameter uncertainties. However, the uncertainties in the calculation of weight percentages of individual components increase due to either or both of two factors: (i) the individual components occur over lower concentration ranges in the mixtures; (ii) spectral differentiation depends on distinguishing small spectral features above the given noise level. The differentiation is more difficult in components with similar Raman spectra such as collagen and elastin. For cholesterol and cholesteryl linoleate, the slopes of the linear correlations between calculated and measured weight percentages, 1.08 and 0.98, respectively, agree with the exact value of 1 to within the uncertainties in the measured Raman weight cross-sections. In the case of cholesteryl oleate, the slope of 0.64 is smaller than the expected value of 1, resulting in a slightly smaller than expected value of 0.81 for total cholesterol ester. The plots also demonstrate that the detection limits for cholesterol, total cholesterol ester, and the individual cholesterol esters are roughly 5% each, which is similar to the total lipid detection limit. this is a consequence of the similar values of the CH₂ band Raman weight cross-sections among cholesterol cholesteryl oleate and cholesteryl linoleate.

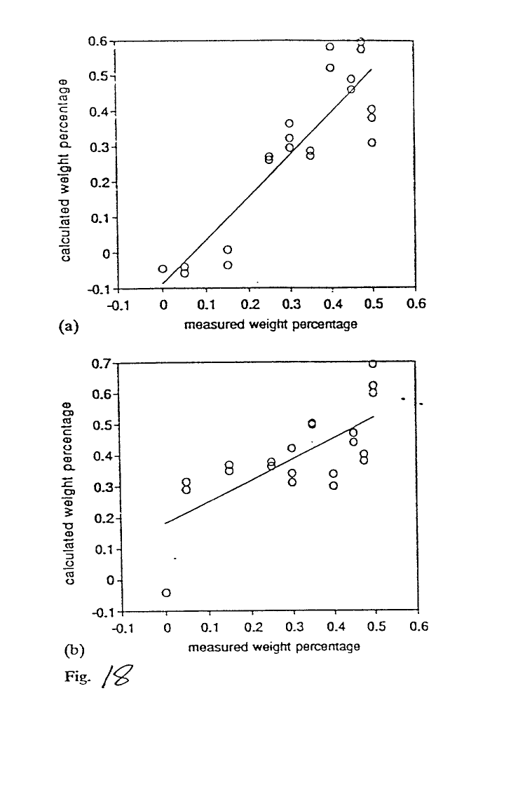

The protein fraction can also be further subdivided into collagen and elastin weight percentages. The calculated weight percentages for collagen and elastin are compared with measured weight percentages in FIG. 18. In these cases, the parameters uncertainties are significantly greater than in the case of the individual lipid components because of the relatively high degree of similarity between the collagen and elastin Raman spectra. These uncertainties obscure the linear correlations between the determined and measured weight percentages, although a linear trend is consistent with the data. The detection limits for collagen and elastin individually are 15-20%, of more than 3 times the 5% detection limits of cholesterol esters.

With the limits of validity of the process established over a wide range of protein and lipid mixtures, we applied the process to Raman spectra collected from intact human aorta. Six biological components were chosen for the initial basis set, rᵢ(ν): collagen (bovine achilles tendon)(FIG. 7b), elastin (bovine neck ligament)(FIG. 7a), cholesterol (FIG. 9a), cholesteryl oleate (FIG. 9c), cholesteryl linoleate (FIG. 9d) and calcium hydroxyapatite (FIG. 11). The carbonated apatite region between 1100 and 1025 cm⁻¹ was excluded in fitting the model to the date, because no sample of this compound is available. Again, the Ch₂ bending band area of each protein and lipid basis spectrum was normalized to unity, as was the symmetric phosphate stretching band in the calcium hydroxyapatite basis spectra. In addition, the Raman spectrum of the buffered saline was included, as it improved the quality of the fits in the 1650 cm⁻¹ region, where the weak O-H bending vibration of water makes a small contribution to the signal. Addition of cholesteryl palmitate as a basis spectrum did not significantly improve the fits of the data.

Measured and calculated FT Raman spectra of typical specimens of normal aorta, atheromatous plaque, and exposed calcified atheromatous plaques are shown in FIGS. 19, 20 and 21 respectively. Residuals of the fits are also plotted in these figures. Weight percentages for each component were computed from the fit coefficients using eqn. (4) and are listed in Table 3. Here, we have adopted the normalization condition that the weight of the organic components (collagen, elastin, cholesterol, cholesteryl oleate, cholesteryl linoleate) for each spectrum sum to 1. In tissue, the weight percentages of these constituents will not in general sum to one due to the presence of the other components in the tissue not detected in the Raman spectra.

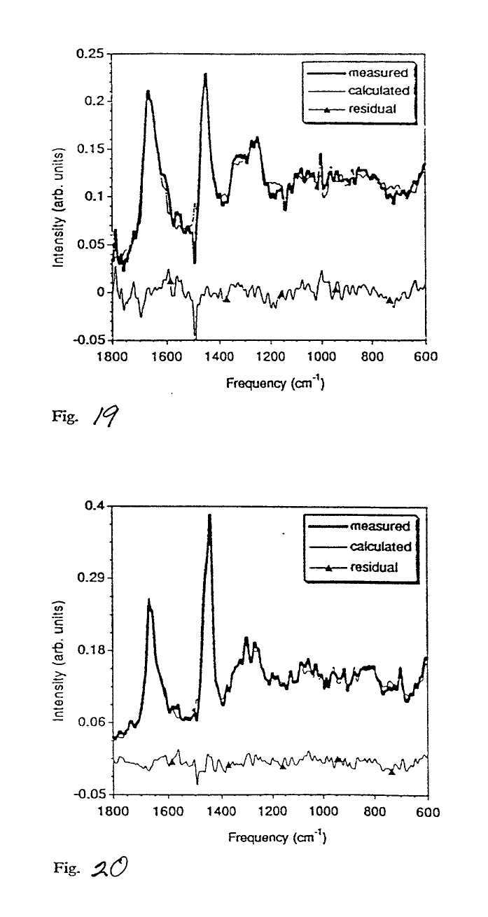

The calculated spectra for both normal aorta (FIG. 19) and atheromatous plaque (FIG. 20) agree quit well with the measured spectra, with only minor deviations from the noise level in the residuals. This suggests that not only does the linear representation hold for tissue, but also that the chosen basis spectra are a reasonable and nearly complete representation of the Raman spectra of the tissue biomolecules to within the spectral signal-to-noise levels.

For example, the calculated collagen:elastin content of the normal aorta spectrum is 31%:62%, while that of the atheromatous plaque is 36%:17%. Also, the normal aorta spectrum yields 6% total cholesterol, the majority being cholesterol ester (oleate), which is consistent with biochemically measured levels. This calculated level is near the detection limit for lipid and is likely significant. In contrast, the computed total cholesterol (cholesterol=cholesterol esters) content for the atheromatous plaque is 47%, with 14% cholesterol, 21% cholesteryl oleate and 12% cholesteryl linoleate.

The two primary bands associated with the deposited calcium salts, 1070 and 960 cm⁻¹, can be incorporated into the procedure with the spectrum of calcium hydroxyapatite. Carbonated apatites exhibit a band at 1070 cm⁻¹ due to the symmetric CC stretching mode. In addition, the width of the 960 cm⁻¹ phosphate stretching band, which in tissue is slightly larger than in pure hydroxyapatite, is known in increase with increasing carbonate substitution in hydroxyapatite. Of the soft tissue components, the procedure calculates 68% collagen, 0% elastin, 9% cholesterol, 4% cholesteryl oleate and 20% cholesteryl linoleate.

In order for Raman spectroscopy of human tissue to become a useful clinical histochemical method, it is desirable one be able to extract quantitative biochemical information from the Raman spectra. NIR FT Raman spectra of human aorta can be used to measure the individual biomolecules which are most prevalent in the tissue, that the signals behave in a linear manner even in a highly scattering environment, and that the signals can be analyzed to extract quantitative or relative quantitative information about the biological composition of atherosclerotic lesions.

The linear representation for extracting the biochemical information can be improved in several ways. The basis spectra can be collected for longer times to increase the signal-to-noise ration and thereby improve the accuracy of the measurement. The basis spectra can be obtained from a large number of samples from human tissue to improve accuracy. There are additional species in arterial tissue which may contribute to the Raman spectra and which can be incorporated into the analytical procedure. For example, in the spectra of calcified plaques, the residuals indicate an additional band at 1070 cm⁻¹, likely due to carbonated apatites. finally, the process can take into account the scattering and inhomogeneities in the tissue. this will enhance measurements for solid structures in the tissue such as calcium hydroxyapatite or cholesterol crystals.

The ability to analyze the mixtures of biological molecules indicates that the process was able to quantitatively determine the character of even complex mixtures with 5-15% accuracy.

The diagnostic utility of NIR and IR Raman spectroscopy, improve on other methods currently utilized in the vascular system for obtaining diagnostic information. Angiography provides information about the length and diameter of a lesion, but cannot supply any biochemical information. angioscopy allows visualization of a lesion which may permit diagnosis of a thrombus or other clearly distinct features, but is limited in the type of data available. Ultrasound can yield information about the density of the material, and thus circumstantially diagnose calcified lesions, but is also very limited in the type of information that can be extracted. Finally, magnetic resonance imaging provides information about the blood flow within the vasculature, but currently has been limited in yielding other chemical information. Thus, Raman measurements are unique in the detail and quantitative nature of the biochemical information it provides.

The information obtained can be used to guide treatment. For example, before deciding on a particular therapy, the physician measures the histochemical information of a lesion such as the percent of cholesterol and cholesterol esters, using Raman spectroscopy. If the lesion contain a large amount of cholesterol, cholesterol lowering drugs might be indicated before proceeding with a more destructive procedure such as a balloon or laser angioplasty. The information provided by the Raman data could be correlated with observations such as the incidence of restenosis after balloon angioplasty, which provides for a better determination of the correct treatment modality. With the Raman technique, biochemical data regarding data regarding the composition of atherosclerotic lesions can be obtained in vivo by insertion of catheters and endoscopes within the vascular system.

The techniques described here are applicable to other tissues and pathologies. For instance, histological detection of malignancies and premalignancies depends in part on determining increases and/or alterations in nuclear material. since Raman spectroscopy is used for probing nucleic acids, this technique can be used to monitor relative nucleic acid concentrations in vivo. Raman spectral differences among normal, benign and malignant tissues can be observed. Raman methods set forth herein provide a method for real-time monitoring of blood components.

TABLE 1
Raman scattering weight cross-sections of different bands from proteins and lipids
typically found in atherosclerotic aorta relative to that of 1 g BaSO₄

| | Vibrational assignment | | | | | | | | | |
|---|---|---|---|---|---|---|---|---|---|---|
| | Ester, C═O | | —C═C— | | CH₂ bend | | C—C stretch | | Sterol ring stretch | |
| | Freq. | Cross- | Freq. | Cross- | Freq. | Cross- | Freq. | Cross- | Freq. | Cross |
| Biological component | (cm⁻¹) | section | (cm⁻¹) | section | (cm⁻¹) | section | (cm⁻¹) | section | (cm⁻¹) | section |
| Collagen | Amide I | 1.00 | — | — | 1450 | 0.72 | — | — | — | — |
| Elastin | Amide I | 1.23 | — | — | 1450 | 0.79 | — | — | — | — |
| Chondroitin sulfate A | Amide | 0.18 | — | — | ˜1400^{a} | 0.58 | — | — | — | — |
| Hyaluronic acid | Amide | 0.58 | — | — | ˜1400^{a} | 0.79 | — | — | — | — |
| Cholesterol | — | — | 1671 | 0.77 | 1440 | 3.19 | — | — | 700 | 0.38 |
| Cholesterol palmitate | 1738 | 0.12 | 1667 | 0.36 | 1440 | 2.70 | 1130 | 0.35 | 700 | 0.13 |
| Cholesteryl oleate | 1738 | 0.12 | 1665 | 1.14 | 1440 | 3.70 | 1140 | 0.17 | 700 | 0.12 |
| Cholesteryl linoleate | 1740 | 0.11 | 1665 | 1.40 | 1440 | 3.02 | 1146 | 0.17 | 700 | 0.12 |
| Palmitic acid | 1737 | 0.52 | — | — | 1442 | 4.66 | 1130 | 0.76 | — | — |
| Tripalmitin | 1745 | 0.41 | — | — | 1440 | 4.32 | 1130 | 0.66 | — | — |

^{a}Calculated for the entire band in the region 1300-1500 cm⁻¹ and probably contains contributions from other modes as well.

TABLE 2
Estimated absolute Raman scattering molecular cross-sections of different bands from lipids
typically found in atherosclerotic aorta^{a}. Units for the absolute cross-section
values are 10⁻³⁰ cm² (molecule · sr)⁻¹

| | Vibrational assignment | | | | | | | | | |
|---|---|---|---|---|---|---|---|---|---|---|
| | Ester, C═O | | —C═C— | | CH² bend | | C—C stretch | | Sterol ring stretch | |
| | Absolute | | Absolute | | Absolute | | Absolute | | Absolute | |
| | cross- | Com- | cross- | Com- | cross- | Com- | cross- | Com- | cross- | Com- |
| Biological component | section | parative^{b} | section | parative^{c} | section | parative^{c} | section | parative^{b} | section | parative^{C} |
| Cholesterol | — | — | 0.67 | 1 | 2.85 | 1 | — | — | 0.34 | 1 |
| Cholesteryl palmitate | 0.17 | 1 | 0.52 | 0.77 | 3.91 | 1.37 | 0.50 | 1 | 0.19 | 0.55 |
| Cholesteryl oleate | 0.18 | 1.06 | 1.73 | 2.58 | 5.58 | 1.96 | 0.26 | 0.52 | 0.18 | 0.53 |
| Cholesteryl linoleate | 0.17 | 1.00 | 2.1 | 3.13 | 4.53 | 1.59 | 0.26 | 0.52 | 0.18 | 0.53 |
| Palmitic acid | — | — | — | — | 2.77 | 0.97 | 0.45 | 0.9 | — | — |
| Tripalmitin | 0.76 | 4.49 | — | — | 8.07 | 2.83 | 1.23 | 2.46 | — | — |

^{a}The Raman cross-section value for S0₄²⁻is 0.54 × 10⁻³⁰ cm² (molecule · sr)⁻¹, corrected for the wavelength dependence [16].
^{b}Molecular cross-sections compared with given band of cholesteryl palmitate.
^{c}Molecular cross-sections comparcd with given band of cholesterol.

TABLE 3
Weight percentages for human aorta calculated from the Raman spectra

| | | | Exposed |
|---|---|---|---|
| Biological component | Normal | Atheromatous | calcification |
| Collagen | 0.31 | 0.35 | 0.68 |
| Elastin | 0.61 | 0.18 | -0.006 |
| Total protein | 0.93 | 0.53 | 0.67 |
| Cholesterol | 0.003 | 0.14 | 0.088 |
| Cholesteryl oleate | 0.064 | 0.21 | 0.036 |
| Cholesteryl linoleate | 0.002 | 0.12 | 0.20 |
| Total lipid² | 0.068 | 0.47 | 0.33 |
| Total cholesteryl ester^{b} | 0.066 | 0.32 | 0.24 |

^{a}Cholesterol + cholesteryl oleate + cholesteryl linoleate.
^{b}Cholesteryl oleate + cholesteryl linoleate.

We claim:

1. A Raman endoscope comprising:
a flexible tubular housing having a first optical waveguide for delivering excitation light from a proximal end of the housing to a distal end of the housing;
a coherent optical fiber bundle positioned within the tubular housing to collect radiation at the distal end of the housing and deliver the collected radiation to the proximal end;
a focal plane array sensor that is optically coupled to the proximal end of the collection bundle to detect radiation having a wavelength in the range of 1-2 microns.

2. The Raman endoscope of claim 1 further comprising a laser optically coupled to the proximal end of the optical waveguide.

3. The Raman endoscope of claim 1 further comprising a broadband light source coupled to the proximal end of the optical waveguide.

4. The Raman endoscope of claim 1 further between the proximal end of the collection bundle and the sensor.

5. The Raman endoscope of claim 1 further comprising a visible light imaging detector coupled to the proximal end of the collection bundle.

6. The Raman endoscope of claim 1 further comprising a plurality of optical fibers for illumination and excitation of an object to be imaged.

7. The Raman endoscope of claim 1 wherein the sensor comprises palladium silicide charge coupled device.

8. The Raman endoscope of claim 1 wherein the sensor comprises a platinum silicide charge coupled device.

9. The Raman endoscope of claim 1 wherein the sensor comprises a Shottky barrier sensor array.

10. A method for Raman imaging of tissue comprising:
inserting an endoscope into a body lumen, the endoscope having an optical waveguide for delivering excitation light through the endoscope and onto tissue to be imaged adjacent a distal end of the endoscope;
directing laser radiation through the optical waveguide and onto the tissue to excite Raman scattered light within the tissue;
detecting the Raman scattered light with a focal plane array sensor to detect radiation having a wavelength in the range of 1-2 microns.

11. The method of claim 10 further comprising coupling a Nd:YAG laser to the optical waveguide.

12. The method of claim 10 further comprising coupling a laser diode emitting light in the range of 800-1200 nm.

13. The method of claim 10 further comprising coupling a broadband light source to the endoscope to illuminate the tissue to be imaged.

14. The method of claim 10 further comprising forming a plurality of images at different infrared wavelengths with the sensor.

15. A Raman endoscope comprising:
an endoscope having an optical fiber extending from a proximal end to a distal end;
a focal plane array sensor at the distal end of the endoscope to detect radiation directed onto the distal end of the endoscope;
a laser optically connected to the optical fiber at the proximal end of the endoscope to irradiate an object to be imaged; and
a memory connected to the sensor for storing an electronic representation of the detected radiation.

16. The Raman endoscope of claim 15 further comprising an additional optical fiber to direct light from a broadband light source onto the object to be imaged.

17. The Raman endoscope of claim 16 further comprising a detector to record a visible image of the object.

18. The Raman endoscope of claim 15 further comprising a data processor and a comparator for comparing images at different wavelengths.

19. The Raman endoscope of claim 15 further comprising an optical system on the distal end of the endoscope.

20. The Raman endoscope of claim 15 further comprising a filter system that filters light directed onto the sensor that selectively transmits light having one or more frequencies selected from the group consisting of 700 cm₋₁, 960 cm⁻¹, 1070 cm⁻¹, 1745 cm⁻¹, 1737 cm⁻¹ and 1440 cm⁻¹.
